(19) United States
(12) Patent Application Publication
(10) Pub. No.: US 2022/0229034 A1
(43) Pub. Date: Jul. 21, 2022

(54) STEERABLE SUBSURFACE GRAIN PROBE

(71) Applicant: Premier Design Innovations LLC, Dawson, NE (US)

(72) Inventors: Rodney R. Heinen, Dawson, NE (US); Shaun J. Kibbe, Sabetha, KS (US); Karin N. Witthar, Valley Center, KS (US); Garrett A. Witthar, Valley Center, KS (US)

(21) Appl. No.: 17/579,794

(22) Filed: Jan. 20, 2022

Related U.S. Application Data
(60) Provisional application No. 63/139,560, filed on Jan. 20, 2021.

(51) Int. Cl. G01N 33/02

(52) U.S. Cl. CPC G01N 33/02

(57) ABSTRACT

A steerable subsurface probe for particulate materials broadly includes a plurality of elongated, helically flighted adjacent bodies, a frame supporting the bodies, a drive assembly, and a cable. The drive assembly is operably coupled with the bodies for selective rotation of the bodies in respective rotational directions and at respective rotational speeds so that the probe may enter a mass of said particulate materials and move beneath the surface of said mass. The cable is attached to the frame, with the end of the cable remote from the frame being at a control location outside of the mass.

### CROSS-REFERENCE TO RELATED APPLICATIONS

This application claims the benefit of U.S. Provisional Application Ser. No. 63/139,560, filed Jan. 20, 2021, entitled STEERABLE SUBSURFACE GRAIN PROBE, which is hereby incorporated in its entirety by reference herein.

### BACKGROUND

### Field

The present invention is broadly concerned with steerable subsurface probes for use in investigating masses of grain or other particulate materials. More particularly, it is concerned with such probes and methods of use thereof, wherein the probes are equipped with two or more adjacent, helically flighted powered bodies which can be actuated to rotate the bodies at respective rotational speeds and respective rotational directions, in order to permit subsurface steering of the probe.

### Description of the Prior Art

A well-known problem in the art of grain handling is referred to as “grain entrapment.” This occurs when grain is being transferred from a bin or the like and, because of undue moisture or other issues, a bridge or internal void occurs within the grain. Typically, an attendant stands on top of the grain and uses a rod or other type of metal implement to break the bridge or determine the location of the void. Unfortunately, this can result in a sudden downward vortex of the grain, which can pull the attendant into the grain, entrapping him. This leads to a number of deaths each year and many other instances where attendants must be rescued. This problem is discussed in a Wikipedia page at https://en.wikipedia.org/wiki/Grain_entrapment. Alternately, there are a number of accessible YouTube videos and other articles which explain the problem of grain entrapment.

The following references are pertinent: US Patent Publications Nos. 2012/0298939 A1, 2015/0346040 A1, and 2020/0-283081 A1; Foreign Patent References Nos. CN105716648A, JPH0919217A, JP 06023284A JP2007006743A, and JP2007082421A; and non-patent literature references: OSHA Directives/Inspection of Grain Handling Facilities, 29 CFR 1910.272 dated Nov. 8, 1996; and “Manual on Grain Management Equipment Maintenance in Silos,” prepared by M. Avun'ana Mushira (FAO Consultant).

The '648 CN reference discloses a spiral propeller device probe connected to a forward/reverse motor 1, the latter transferring power to the auger probe via a Bowden cable. The direction of rotation of the probe causes it to travel beneath the grain surface, or to withdraw therefrom. However, the disclosed probe cannot be steered within the grain because the motion thereof is essentially linear, either downwardly through the grain, or upwardly toward the surface of the grain.

There is accordingly a need in the art for improved steerable subsurface devices which can be inserted into a mass of grain or other particulate materials (e.g., snow) and then steered from a control location outside of the mass, in order to determine the characteristics of the mass, all without the need to stand on the surface of the mass.

This background discussion is intended to provide information related to the present invention which is not necessarily prior art.

### SUMMARY

The following brief summary is provided to indicate the nature of the subject matter disclosed herein. While certain aspects of the present invention are described below, the summary is not intended to limit the scope of the present invention.

Aspects of the present invention provide solutions to the problems outlined above, in the form of a steerable subsurface probe for particulate materials, such as grains.

A first aspect of the present invention concerns a steerable subsurface probe for particulate materials that broadly includes a plurality of elongated, helically flighted adjacent bodies, a frame supporting the bodies, a drive assembly, and a cable. The drive assembly is operably coupled with the bodies for selective rotation of the bodies in respective rotational directions and at respective rotational speeds so that the probe may enter a mass of said particulate materials and move beneath the surface of said mass. The cable is attached to the frame, with the end of the cable remote from the frame being at a control location outside of the mass.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWINGS

FIG. 1 is a fragmentary rear perspective view of a subsurface probe constructed in accordance with a first embodiment of the present invention, with the probe including a frame assembly, flighted bodies, a cable, and struts attaching the cable to the frame assembly;

FIG. 2 is a side elevational view of the probe depicted in FIG. 1, with each of the flighted bodies including a central section, a conical nose section, and a conical rearward end section;

FIG. 3 is a front elevation view of the probe depicted in FIGS. 1 and 2;

FIG. 4 is a bottom view of the probe depicted in FIGS. 1-3;

FIG. 5 is a cross-sectional view of the probe taken along line 5-5 in FIG. 4, showing a drive assembly with transmission elements received by each of the flighted bodies;

FIG. 6 is a cross-sectional view of the probe taken along line 6-6 in FIG. 4, showing sections of each flighted body drivingly attached to and supported by a respective longitudinal shaft and powered by a corresponding electric motor;

FIG. 7 is a fragmentary perspective view of the probe similar to that of FIG. 1, but with portions of the rotatable, flighted bodies being removed to illustrate structure located within the flighted bodies;

FIG. 8 is a fragmentary side elevation view of the probe depicted in FIGS. 1-7, depicting the drive assembly for powering the flighted bodies;

FIG. 9 is a cross-sectional view of the probe taken along line 9-9 in FIG. 8, showing transmission elements received by each of the flighted bodies;

FIG. 10 is a cross-sectional view of the probe taken along line 10-10 in FIG. 8;

FIG. 11 is an enlarged fragmentary perspective view of the probe shown in FIGS. 1-10, showing the drive assembly associated with one of the flighted bodies;

FIG. 12 is a fragmentary rear perspective view of a subsurface probe constructed in accordance with a second embodiment of the present invention, with the probe including a frame assembly, flighted bodies, a cable, and struts attaching the cable to the frame assembly;

FIG. 13 is a cross-sectional view of the probe depicted in FIG. 12, showing a drive assembly with transmission elements received by each of the flighted bodies, and further showing access doors incorporated into the nose sections of flighted bodies for collecting material samples from the particulate mass; and

FIG. 14 is a fragmentary rear perspective view of a subsurface probe constructed in accordance with a third embodiment of the present invention, with the probe including a frame assembly, flighted bodies, and drive cables extending from a remote station to respective ones of the flighted bodies.

The drawing figures do not limit the present invention to the specific embodiments disclosed and described herein. While the drawings do not necessarily provide exact dimensions or tolerances for the illustrated components or structures, the drawings, not including any purely schematic drawings, are to scale with respect to the relationships between the components of the structures illustrated therein.

### DETAILED DESCRIPTION OF THE PREFERRED EMBODIMENTS

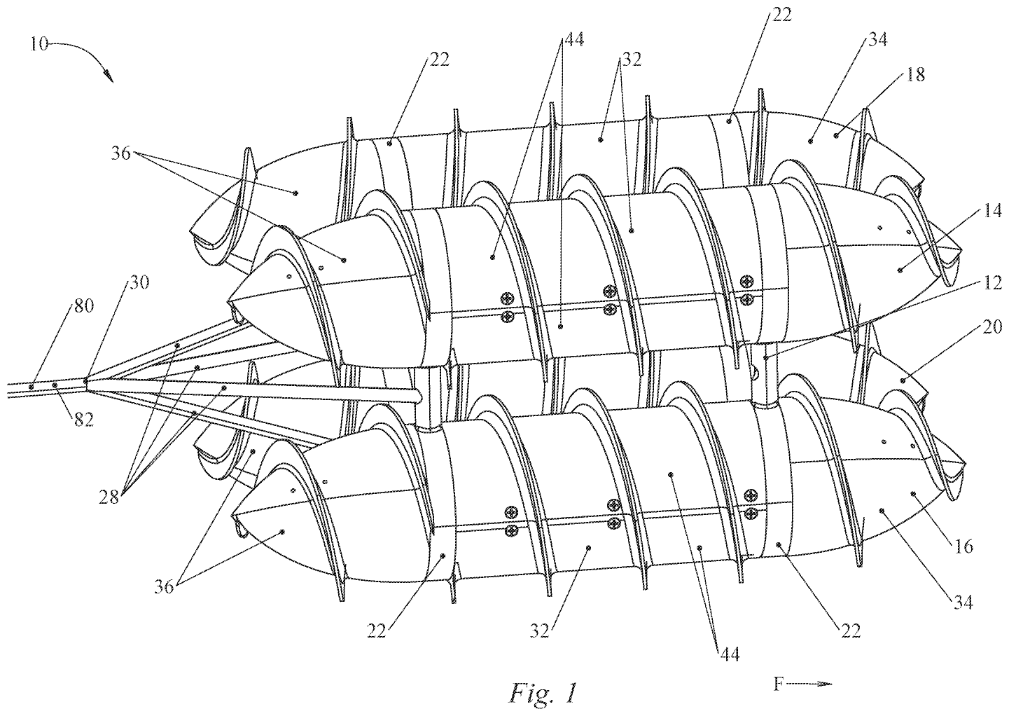

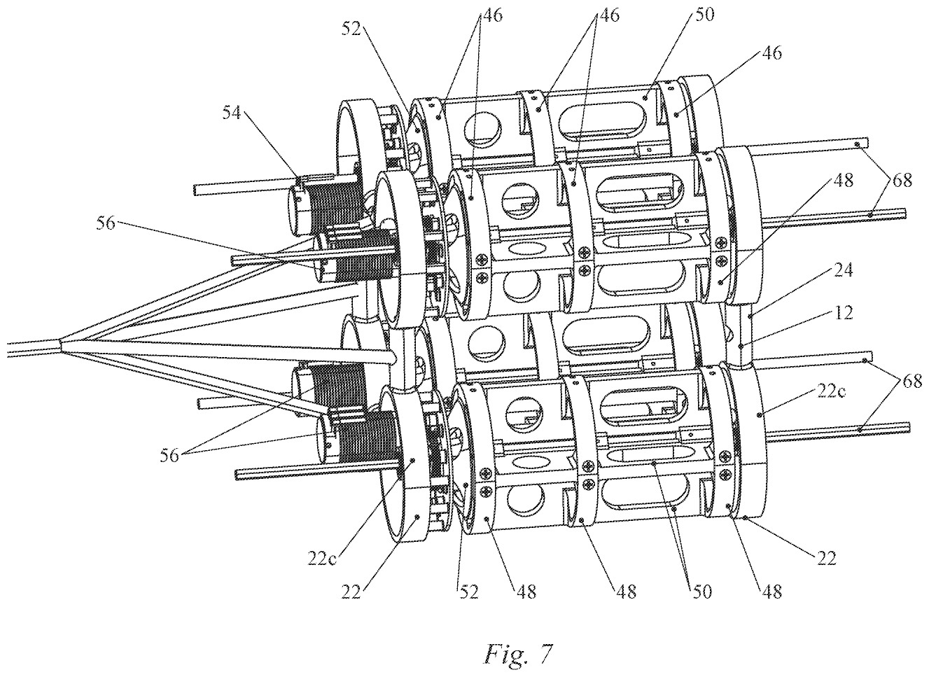

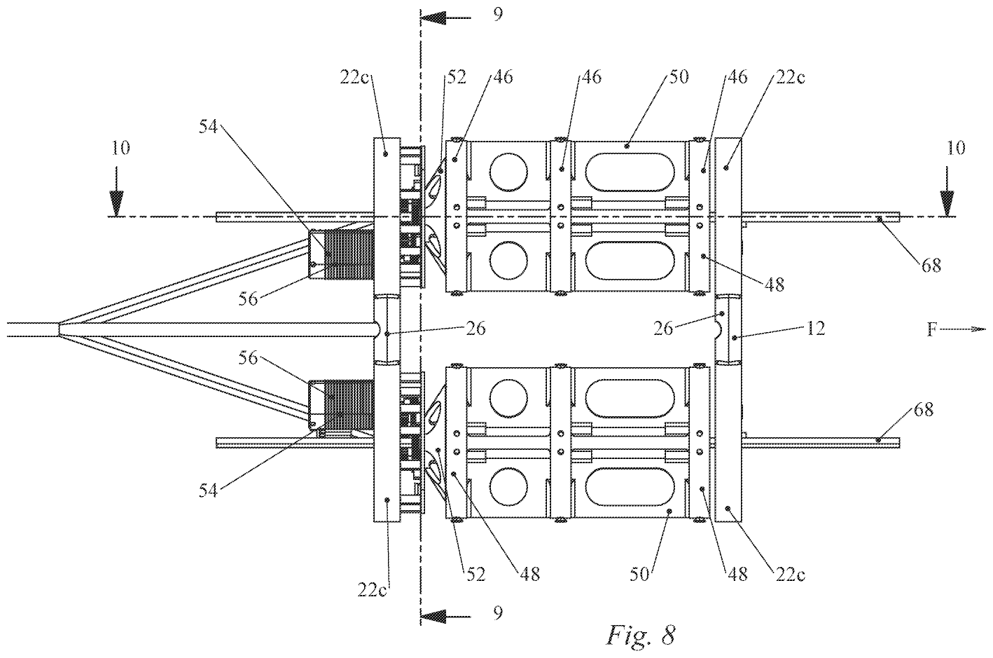

Turning initially to FIGS. 1-11, a steerable, subsurface particulate probe 10 generally includes a rigid frame assembly 12, and a total of four elongated, axially rotatable, helically flighted bodies 14,16,18,20 supported by the frame assembly 12. The probe 10 is designed to be placed within a mass of grain or other particulate material (not shown), and to be activated to pass under the surface of the particulate mass for inspection purposes. To this end, the probe 10 is steerable from a remote control location outside of the particulate mass to facilitate inspection thereof. As will be explained, the probe 10 is configured to be advanced in a forward direction F or in an opposite rearward direction.

In preferred embodiments, probe 10 is configured to be advanced into and through a mass of grain, such as wheat, corn, soybeans, milo, etc. It will also be appreciated that probe 10 may be used to inspect a particulate mass that includes other types of particulate material. Alternative particulate materials may include synthetic resin, sand, salt, flour, wood, paper, metal, etc. It will also be understood that the particulate mass may include one or more of various particulate sizes. Furthermore, a particular mass may be identified as including fine powders, granules, pellets, grains, kernels, berries, seeds, beans, beads, pills, tablets, etc.

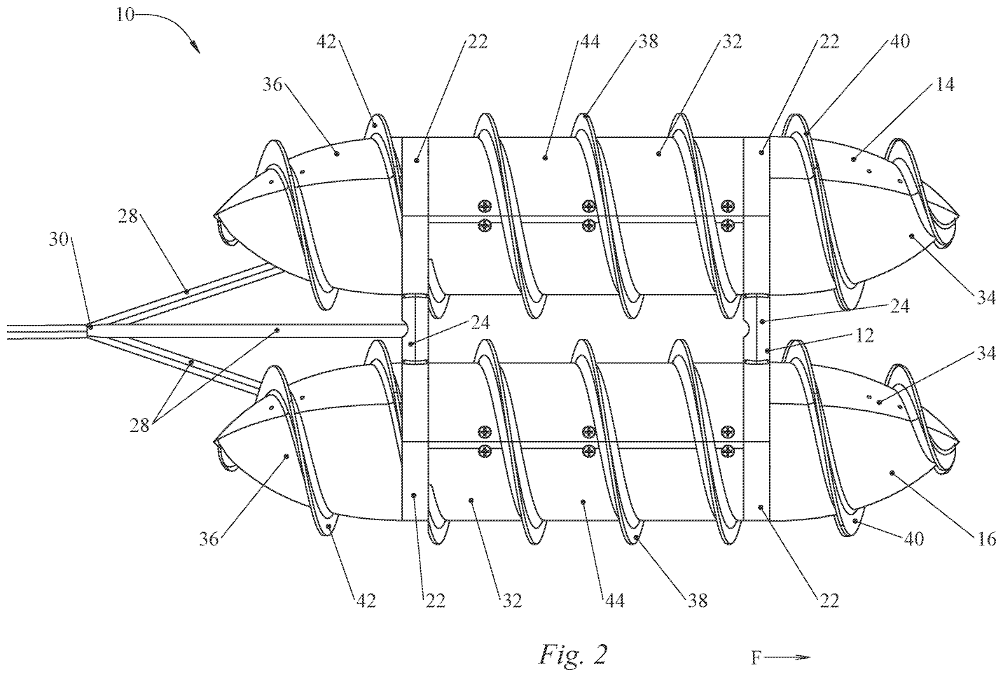

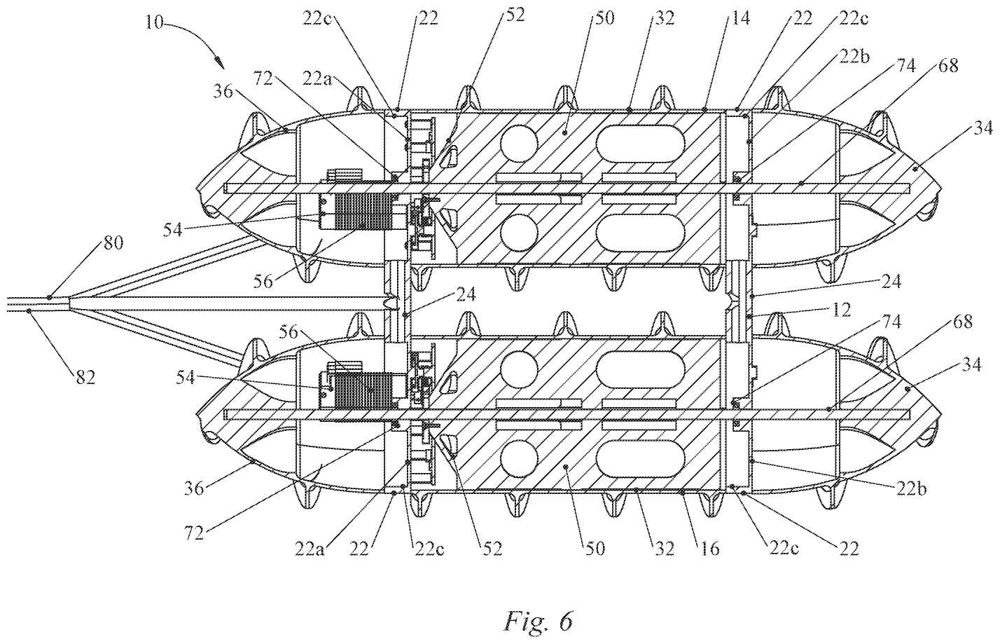

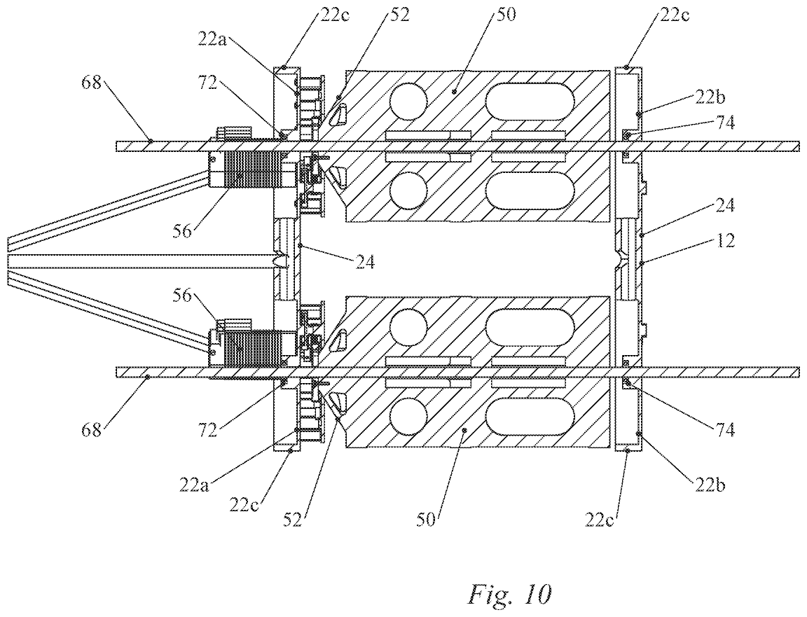

In more detail, the frame assembly 12 includes a total of eight identical bearing supports 22, which are arranged in spaced apart aligned pairs, as best seen in FIGS. 1 and 2. The bearing supports 22 includes wall 22a,22b and circular outer rings 22c (see FIGS. 6 and 10). Each pair of aligned supports 22 rotatably supports a respective one of the flighted bodies 14,16,18,20 to permit smooth, low-friction, rotation of the body relative to the frame assembly 12.

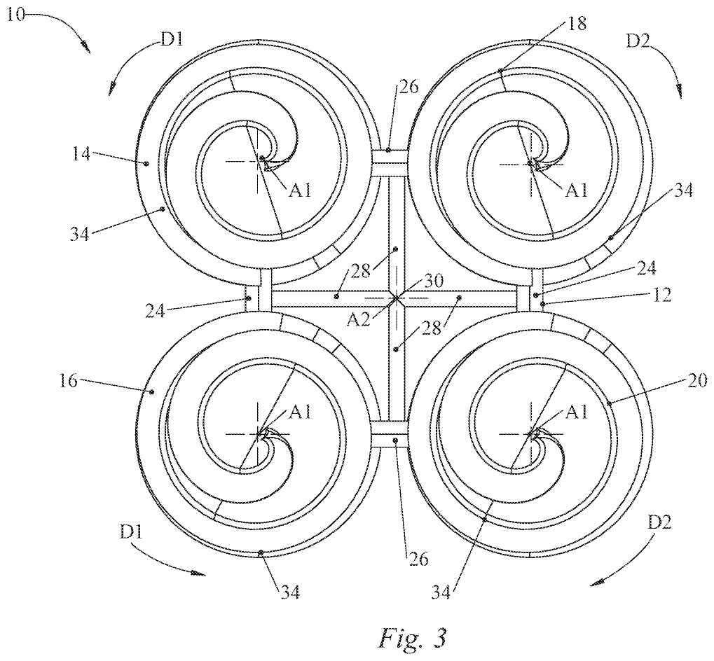

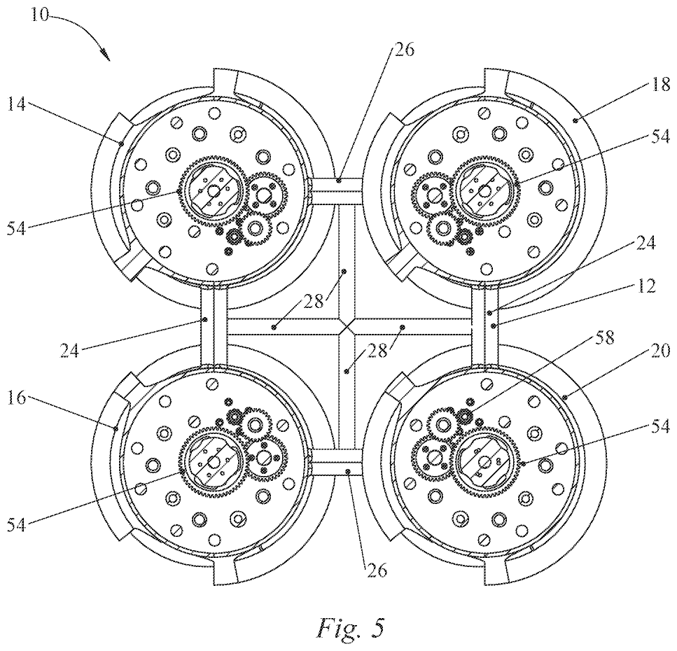

The bearing supports 22 are interconnected by means of upright tubular couplers 24 and laterally extending tubular couplers 26 (see FIGS. 3 and 5). In addition, the overall frame 12 includes a total of four rearwardly extending tubular struts 28, which are respectively secured to the rearward couplers 24, 26 and join to form an apex 30 (see FIGS. 1-3).

The depicted frame configuration has a generally rigid construction and is preferred for rotatably supporting the bodies 14,16,18,20 during operation of the grain probe 10. However, for at least certain aspects of the present invention, embodiments of the grain probe may include an alternative frame construction for supporting one or more bodies. For instance, the frame may include one or more alternative rotational supports, additionally or alternatively to bearing rings, for rotatably supporting the flighted bodies. Alternative rotational supports may be located at alternative locations along the length of the flighted bodies. For instance, an alternative frame may include supports located at or adjacent to the forwardmost tip and/or the rearmost tip of the flighted body.

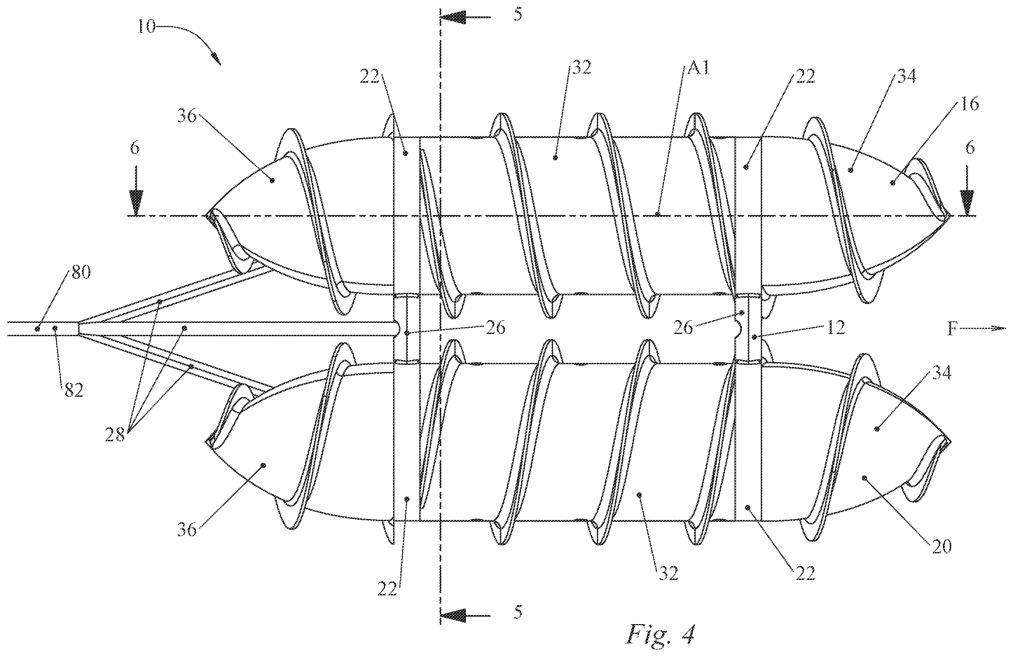

In the depicted embodiment, flighted bodies 14,16,18,20 have a similar construction and are each configured to drive the probe 10 through a mass of particulate material. Each body 14,16,18,20 includes an elongated, central tubular section 32, a tapered, conical forward nose section 34, and a tapered, conical, rearward end section 36. As seen, the central section 32 is equipped with outwardly extending helical fighting 38, whereas the conical sections 34 and 36 are likewise equipped with helical fighting 40 and 42, respectively. Sections 32,34,36 of each body 14,16,18,20 are configured to rotate about a corresponding longitudinal body axis A1 (see FIGS. 3 and 4).

Preferably, a right pair of bodies 14,16 are juxtaposed with a left pair of bodies 18,20. Further, the right pair of bodies 14,16 have helical fighting that is of opposite hand relative to the helical fighting of the left pair of bodies 18,20. In the depicted embodiment, helical flighting of the right pair of bodies 14,16 comprises a right-handed fighting, relative to the forward direction F, while the helical flighting of the left pair of bodies 18,20 comprises a left-handed fighting, relative to the forward direction F.

Thus, to advance in a forward direction, each of the right pair of bodies 14,16 is rotated in a counterclockwise direction D1 when viewed from the front, and each of the left pair of bodies 18,20 is rotated in a clockwise direction D2 when viewed from the front (see FIG. 3). Conversely, to advance in a rearward direction, each of the right pair of bodies 14,16 is rotated in a clockwise direction when viewed from the front, while each of the left pair of bodies 18,20 is rotated in a counterclockwise direction when viewed from the front.

The opposite-handed flighting configuration of the right and left pairs of bodies permits the probe 10 to be advanced forwardly and rearwardly without rolling (that is, rotating) of the entire probe 10 about a longitudinal probe axis A2 (see FIG. 3) thereof. Although the disclosed fighting configuration preferably restricts roll movement of the probe 10 during at least certain operational movements, roll movement of the probe may be provided in other instances.

It is also within the scope of certain aspects of the present invention for one or more flighted bodies to have an alternative flighting configuration (e.g., to restrict rolling of the probe 10 about the longitudinal axis). For instance, an alternative probe may include an upper pair of flighted bodies with helical flighting that is of opposite hand relative to the helical fighting of a lower pair of bodies (as depicted in the incorporated '560 application).

In use, the bodies 14,16,18,20 may be operated to initiate lateral turning of the probe 10. For instance, probe 10 may be turned to the right when all bodies 14,16,18,20 are advanced in the forward direction by rotating the left pair of bodies 18,20 at a faster speed than the right pair of bodies 14,16. The probe 10 may also be turned to the right by advancing the left pair of bodies 18,20 in the forward direction while advancing the right pair of bodies 14,16 in the rearward direction. Yet further, the probe 10 may be turned to the right when all bodies 14,16,18,20 are advanced rearwardly by rotating the right pair of bodies 14,16 at a faster speed than the left pair of bodies 18,20.

Similarly, the probe 10 may be turned to the left when all bodies 14,16,18,20 are advanced in the forward direction by rotating the right pair of bodies 14,16 at a faster speed than the left pair of bodies 18,20. The probe 10 may also be turned to the left by advancing the right pair of bodies 14,16 in the forward direction while advancing the left pair of bodies 18,20 in the rearward direction. Yet further, the probe 10 may be turned to the left when all bodies 14,16,18,20 are advanced rearwardly by rotating the left pair of bodies 18,20 at a faster speed than the right pair of bodies 14,16.

Bodies 14,16,18,20 may also be pitched upwardly or downwardly to initiate climbing or descent of the probe 10. For example, probe 10 may be pitched upwardly when all bodies 14,16,18,20 are advanced in the forward direction by rotating the upper pair of bodies 14,18 at a slower speed than the lower pair of bodies 16,20. The probe 10 may also be pitched upwardly by advancing the lower pair of bodies 16,20 in the forward direction while advancing the upper pair of bodies 14,18 in the rearward direction. Yet further, the probe 10 may be pitched upwardly when all bodies 14,16,18,20 are advanced rearwardly by rotating the lower pair of bodies 16,20 at a slower speed than the upper pair of bodies 14,18.

Probe 10 may instead be pitched downwardly when all bodies 14,16,18,20 are advanced in the forward direction by rotating the upper pair of bodies 14,18 at a faster speed than the lower pair of bodies 16,20. The probe 10 may also be pitched downwardly by advancing the lower pair of bodies 16,20 in the rearward direction while advancing the upper pair of bodies 14,18 in the forward direction. Yet further, the probe 10 may be pitched downwardly when all bodies 14,16,18,20 are advanced rearwardly by rotating the lower pair of bodies 16,20 at a faster speed than the upper pair of bodies 14,18.

In the depicted embodiment, each fighting 38,40,42 comprises a helically shaped flighting structure that is unitary and extends continuously from end to end along the respective section. Each fighting 38,40,42 comprises a single-start “thread” configuration with a single helical lip or “thread.”

As used herein, the term “helical” includes a structure with a smooth helical shape or a structure with an approximate helical shape, where the shape includes one or more deviations from a smooth helical shape (e.g., where a deviation may include one or more of a linear element, sharp angle, break, discontinuity, convex scallop, concave scallop, etc.).

However, one or more of the sections may be configured with flighting that includes multiple flighting elements. For instance, one or more of the flighting may include a multi-start “thread” configuration with two (2) or more helical lips or “threads” that generally extend parallel to one another.

For certain aspects of the present invention, alternative flighted bodies may include fighting that does not extend continuously from end to end of the corresponding section. Such alternative embodiments may include a series of fighting segments that are longitudinally spaced apart from one another. Yet further, alternative embodiments may include a section with fighting that is not helical (e.g., flighting segments in a non-helical arrangement). It is also within the scope of certain aspects of the present invention for one or more sections of the bodies to be devoid of flighting.

Preferably, fighting 38 is integrally provided as part of the tubular section 32 (see FIG. 2). Similarly, flighting 40,42 is preferably integrally provided as part of respective conical sections 34,36 (see FIG. 2). However, it will be appreciated that one or more segments of flighting may be detachable from a remainder of a respective section.

In the illustrated embodiment, tubular section 32 includes a pair of removable, curved panel segments 44 (see FIG. 1) that are removable to permit access to the interior of the body 14. Although flighted bodies 14,16,18,20 are preferably similar to one another, other embodiments may have one or more flighted bodies that have an alternative construction.

The depicted probe embodiment preferably has two (2) pairs of flighted bodies. However, for certain aspects of the present invention, alternative probe embodiments may include fewer than four (4) bodies or greater than four (4) bodies. For example, an alternative probe may have a single pair of flighted bodies with flighting that are opposed to one another. It will be appreciated that such an embodiment may include a steering mechanism other than the bodies for controlling the direction of probe advancement.

Attention is next directed to FIGS. 5-11, which illustrate internal components received by the bodies 14,16,18,20. As illustrated, the central section 32 is internally supported by curved rings 46 and 48 secured to the inner surface of section 32. The central section 32 is also supported via longitudinal braces 50 and end plate 52.

Bearing supports 22 are positioned fore and aft of the central section 32 and are slidably engaged with the central section 32. The bearing supports 22 are also slidably engaged with and support the conical sections 34 and 36.

Drive assembly 54 is operably coupled with the bodies 14,16,18,20 for selective rotation of the bodies 14,16,18,20 in respective rotational directions and at respective rotational speeds so that the probe 10 may enter the particulate mass and move beneath the surface thereof.

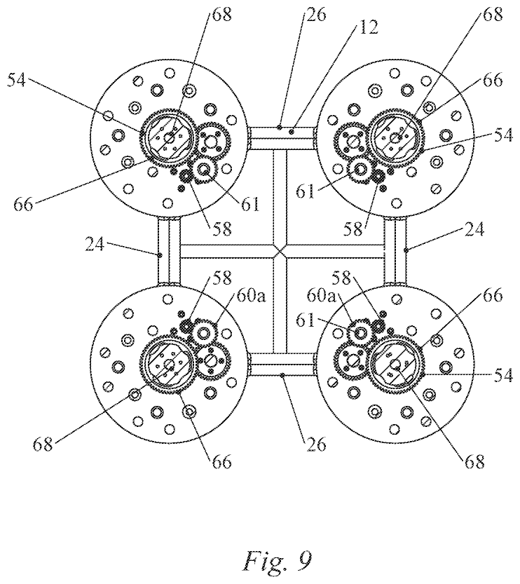

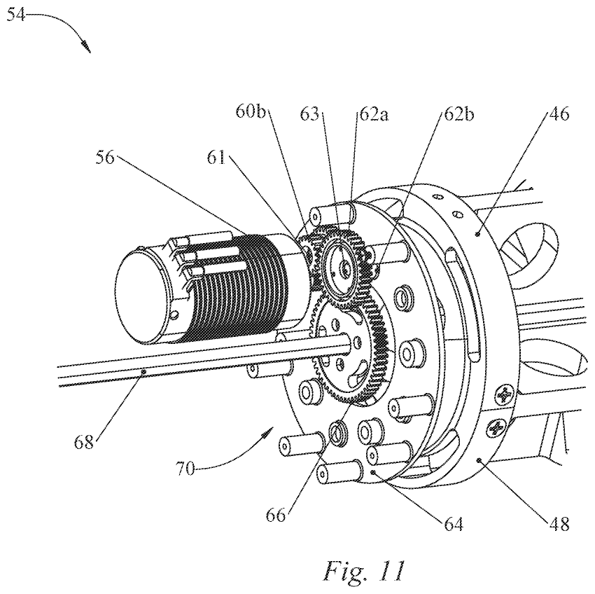

Elements of the drive assembly 54 are preferably located within respective bodies 14,16,18,20 to effect axial rotation thereof. The drive assembly 54 includes reversible DC electric motors 56 located in respective bodies 14,16,18,20. Each motor 56 is secured to a corresponding wall 22a and has an output shaft that drivingly receives and rotates with a spur gear 58 (see FIG. 9). Intermediate gear 60a is meshed with spur gear 58 and is rotatably supported on a shaft 61 with another intermediate gear 60b, so that the gears 60a,60b rotate with one another (see FIGS. 9 and 11). The intermediate gear 60b is meshed with a secondary spur gear 62a (see FIG. 11). The secondary spur gear 62a is rotatably supported on a shaft 63 with another secondary spur gear 62b (see FIG. 11), so that the gears 62a,62b rotate with one another. Gears 60a,60b,62a,62b and shafts 61,63 are rotatably supported by frame 64 and wall 22a.

The spur gear 62b is in turn meshed with a primary drive gear 66 (see FIGS. 9 and 11). A shaft 68 is fixed to primary drive gear 66 so that the drive gear 66 and shaft 68 rotate with one another (see FIGS. 9 and 11). The rearmost end of shaft 68 extends through conical end section 36 and is attached at the apex thereof (see FIG. 6). The shaft 68 also extends axially through the wall 22a and is rotatably supported relative to the wall 22a by a bearing 72 (see FIG. 6). Shaft 68 also extends through the wall 22b and is rotatably supported relative to wall 22b by a bearing 74 for attachment to the nose section 34 (see FIG. 6).

The illustrated gears 58,60a,60b,62a,62b,66 are cooperatively provided as part of a preferred transmission 70 (see FIG. 11) that transfers power from a respective motor 56 to a corresponding one of the shafts 68 (or another part of the respective flighted body). It is also within the scope of certain aspects of the present invention for embodiments of the drive assembly to include an alternative transmission. For instance, alternative transmission elements may include, but are not limited to, one or more alternative gears, a chain-and-sprocket drive, a belt-and-pulley drive, one or more frictionally-engaged drive elements, or combinations thereof

While the use of electric motor 56 is preferred, one or more of the flighted bodies may be driven by an alternative powered motor. For instance, such alternative motors may include a hydraulic motor or a pneumatic motor. When using a fluid-powered motor, it will be appreciated that a supply of pressurized fluid may be supplied to the motor via a flexible conduit (not shown), which may be received within the cable, supported alongside the cable and attached thereto, or supported independently of the cable.

Each flighted body 14,16,18,20 preferably receives and is driven by a respective motor 56. In various embodiment of the probe, one or more motors used to drive a corresponding flighted body may be located externally of the flighted body, as will be shown in a subsequent embodiment. Furthermore, an alternative probe may have multiple flighted bodies that are driven by a common motor.

An elongated cable 80 includes an elongate, flexible tubular conduit 82 and flexible electrical leads (not shown) contained within the conduit 82. The cable 80 is secured to apex 30 and extends to the control location outside of the particulate mass. Thus, the requisite electrical leads for the four motors 56 extend through the conduit 82, struts 28, and couplers 24 for attachment to the individual motors 56.

In alternative embodiments, the cable may be alternatively configured without departing from the scope of the present invention. For instance, one or more alternative cables may include hydraulic or pneumatic lines to provide a pressurized fluid flow (such as pressurized hydraulic or pneumatic flow) to power a hydraulic or pneumatic motor.

For certain aspects of the present invention, alternative probe embodiments may be provided with an onboard source of power (e.g., a rechargeable battery and/or non-rechargeable battery) to provide power to the drive assembly or other probe components.

It is also within the scope of certain aspects of the present invention for probe embodiments to be devoid of a cable that interconnects a control station and the probe. For instance, the drive assembly may communicate wirelessly with the control station in certain embodiments.

It will be observed that the fighting forming a part of the right pair of bodies 14 and 16 are of opposite hand as compared with the fighting of the left pair of bodies 18 and 20. Again, in this embodiment, the flighting of right bodies 14,16 is of right hand, whereas the fighting of left bodies 18,20 is of left hand.

In order to control and steer the probe 10, suitable actuating apparatus is provided at the end of cable 80 at a control station. This apparatus may take a variety of forms. For example, one or more joysticks may be secured to the motor leads in order to individually control the rate and direction of operation of the motors 56. In other instances, a wireless control may be used, which may eliminate the need for electrical leads altogether.

In use, the probe 10 is placed at least partly below the surface of the particulate mass to be inspected, and the individual motors 56 are actuated to move the probe downwardly into the particulate mass, and then laterally as needed for inspection purposes. For advancement in the forward direction, motors 56 are preferably operated so that the rotational speed of the flighted bodies preferably ranges from about 30 revolutions per minute (rpm) to about 125 rpm, and more preferably, ranges from about 70 rpm to about 110 rpm. If it is desired to steer the probe left or right, the motors 56 are differentially actuated to turn the probe, as described above. Similarly, if the probe needs to be pitched up or down, the motors 56 are differentially actuated to pitch the probe as described above. At the end of an inspection, the motors 56 may be reversed, causing the probe to return to the surface of the particulate mass. In the event that the probe 10 becomes inoperative within a particulate mass, the cable 80 may be used to rescue the probe by pulling the probe to the surface of the mass.

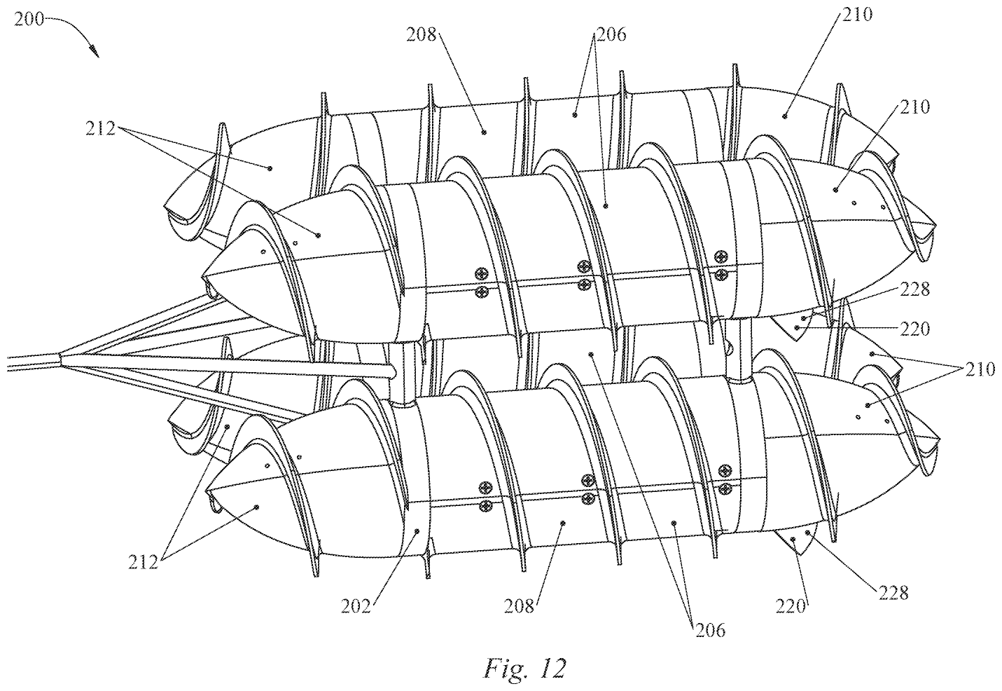

Turning to FIGS. 12-14, alternative preferred embodiments of the present invention are depicted. For the sake of brevity, the remaining description will focus primarily on the differences of these alternative embodiments from the preferred embodiment described above.

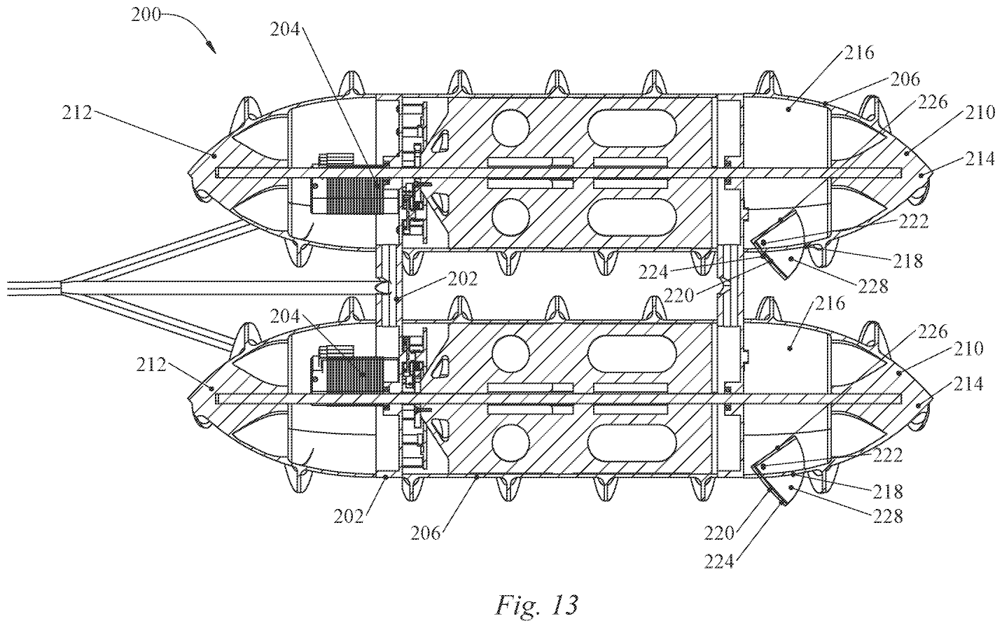

Initially turning to FIGS. 12 and 13, an alternative probe 200 is constructed in accordance with a second embodiment of the present invention. The probe 200 preferably includes, among other things, a frame assembly 202, a drive assembly 204, and alternative flighted bodies 206. The flighted bodies 206 are rotatably supported by the frame assembly 202 and powered by the drive assembly 204.

Flighted bodies 206 each preferably include an elongated, central tubular section 208, an alternative nose section 210, and a rearward end section 212. Nose section 210 includes a conical housing 214 that defines an interior chamber 216 and presents an access opening 218. Nose section 210 also preferably includes a pivotal door 220 that is pivotally mounted to the housing 214 at a pivot joint 222. Door 220 is generally unitary and rigid and includes panels 224,226 connected by opposite side walls 228.

Door 220 is preferably shiftable between a closed position (not shown), in which the panel 224 of door 220 covers the access opening 218, and an open position (see FIGS. 12 and 13), in which the panel 224 of door 220 is opened outwardly and permits particulate material to flow into the chamber 216. The flighted body 206 also preferably includes a motor (not shown) to shift the door 220 between open and closed positions. The door 220 is preferably opened to permit collection of a sample of particulate material as the probe 200 is advanced through a particulate mass. The door 220 may be selectively closed once the material sample has been collected.

Although the depicted sample collection device is preferred, embodiments of the probe may include an alternative device for collecting a sample of the particulate material. For instance, an alternative rearward end section or an alternative central tubular section may include a door and a chamber for collecting one or more material samples. Yet further, alternative probe embodiments may include a sample collection device that is not provided as part of the flighted bodies.

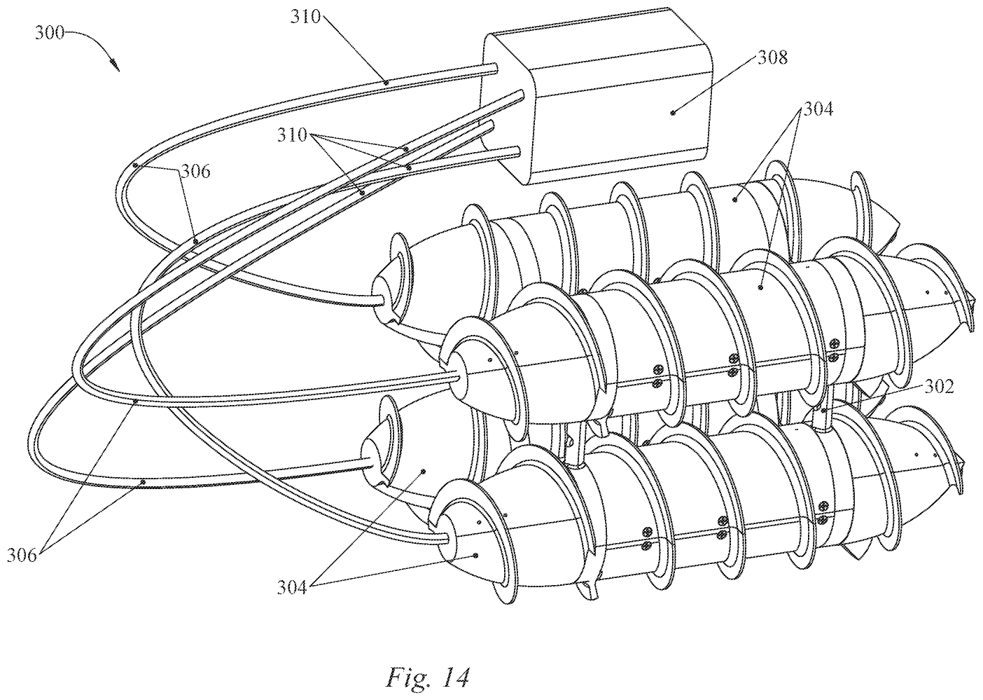

Turning to FIG. 14, an alternative probe 300 is constructed in accordance with a third embodiment of the present invention. Probe 300 preferably includes, among other things, a frame assembly 302, flighted bodies 304, alternative flexible cables 306, and remote station 308. Each cable 306 includes an outer sheath 310 and a flexible internal drive line (not shown) that rotates within the sheath 310. Each drive line is rotatably powered by a reversible motor (not shown) located within the remote station 308. Each drive line is also drivingly attached to the shaft associated with each of the flighted bodies 304, so that motor rotation causes rotation of the respective flighted body 304.

Additional advantages of the various embodiments of the invention will be apparent to those skilled in the art upon review of the disclosure herein. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

As used herein, the term “includes” may refer to an item that includes something as a part thereof or is entirely made up of that something.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

The preferred forms of the invention described above are to be used as illustration only, and should not be utilized in a limiting sense in interpreting the scope of the present invention.

Obvious modifications to the exemplary embodiments, as hereinabove set forth, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

We claim:

1. A steerable subsurface probe for particulate materials, comprising:
a plurality of elongated, helically flighted adjacent bodies;
a frame supporting said bodies;
a drive assembly operably coupled with the bodies for selective rotation of the bodies in respective rotational directions and at respective rotational speeds so that the probe may enter a mass of said particulate materials and move beneath the surface of said mass; and
a cable attached to said frame, with the end of the cable remote from the frame being at a control location outside of said mass.

2. The probe of claim 1, there being four of said bodies arranged to present a right pair of rotational bodies, and a left pair of rotational bodies juxtaposed with said right pair of rotational bodies, the helical fighting of the right and left pairs of rotational bodies being of opposite hand.

3. The probe of claim 1, said drive assembly comprising a motor within each of said bodies in order to selectively rotate each body.

4. The probe of claim 3, said cable being tubular, said motors being electrical motors, there being electrical leads within said cable and operably coupled with said motors in order to permit selective energization of the motors from said control location.

5. The probe of claim 1, each of said bodies having helically flighted conical ends.

6. A method of examining a mass of particulate material, comprising the step of inserting the probe of claim 1 into said mass, and selectively actuating said drive assembly to cause the probe to move within said mass beneath the surface thereof.

7. The method of claim 6, including the step of selectively rotating said bodies at individual rotational speeds and rotational directions, in order to steer the probe within said mass.
